(19) United States
(12) Patent Application Publication
(10) Pub. No.: US 2022/0125878 A1
(43) Pub. Date: Apr. 28, 2022

(54) Compositions and Methods for Preventing the Interaction Between SARS-COV-2 and L-Sign

(71) Applicant: Oklahoma Medical Research Foundation, Oklahoma City, OK (US)

(72) Inventors: Lijun Xia, Edmond, OK (US); Yuji Kondo, Oklahoma City, OK (US)

(21) Appl. No.: 17/506,370

(22) Filed: Oct. 20, 2021

Related U.S. Application Data
(60) Provisional application No. 63/105,073, filed on Oct. 23, 2020.

(51) Int. Cl. A61K 38/17; C07K 16/28; A61K 31/715

(52) U.S. Cl. CPC A61K 38/178; A61K 31/715; C07K 16/2851

(57) ABSTRACT

The present invention includes methods for treating and/or preventing a viral or virally-induced tissue dysfunction or failure in a patient comprising administering an inhibitor of SARS-CoV-2 binding to L-SIGN in an amount effective to treat the viral or virally-induced tissue dysfunction or failure of cells do not express ACE2.

### CROSS-REFERENCE TO RELATED APPLICATIONS

This application is a non-provisional patent application of and claims priority to U.S. provisional patent application Ser. No. 63/105,073 filed on Oct. 23, 2020, the contents of which are incorporated by reference in its entirety.

### STATEMENT OF FEDERALLY FUNDED RESEARCH

This invention was made with government support under 1R01HL153728-01, and 5R01HL128390-04 awarded by the National Institutes of Health. The government has certain rights in the invention.

### TECHNICAL FIELD OF THE INVENTION

The present invention relates in general to the field of agents for the treatment of COVID19, and more particularly, to compositions and methods for preventing the interaction between SARS-CoV-2 and L-SIGN .

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2020, is named ______.txt and is ______ bytes in size.

### BACKGROUND OF THE INVENTION

Without limiting the scope of the invention, its background is described in connection with coronaviruses.

Severe acute respiratory syndrome coronavirus-2 (SARS-CoV-2) infection causes coronavirus disease 2019 (COVID-19). Despite unprecedented worldwide effort, COVID-19 remains a pandemic with global deaths approaching 1 million.¹ While the majority of the infected individuals are asymptomatic, symptomatic patients have a wide spectrum of clinical manifestations, ranging from viral pneumonia and acute respiratory distress syndrome to extrapulmonary injuries to the heart, kidney, liver, and other organs. Multiorgan involvement is an important risk factor for severity and mortality.

SARS-CoV-2 is an enveloped, positive-sense RNA member of the Betacoronavirus genus known to infect humans. Angiotensin converting enzyme 2 (ACE2) is the sole known receptor mediating the entry of SARS-CoV-2 into host cells^{2,3} ACE2 expression has been found on alveolar epithelial type II cells of the lung and on nasal epithelial cells, renal tubular cells, and intestinal epithelial cells⁴, which is thought to contribute to virus-mediated injury of these organs. Up to 71.4% of critically ill COVID-19 patients have thrombosis and coagulopathy, often associated with poor prognosis.^{5,6} Multiple studies, including a multicenter prospective cohort study, indicate that coagulopathy and thrombosis in COVID-19 patients are characterized by increased plasma levels of D-dimer (fibrin degradation product), von Willebrand factor (VWF), and coagulation factor VIII (FVIII)^{7,8}, which is thought to be caused by SARS-CoV-2-induced inflammatory responses or sepsis.^{2,7,8} Markers of endothelial activation such as elevated levels of soluble P-selectin, CD40L, and thrombomodulin have been documented in the plasma of patients with severe COVID-19⁷. However, recent single-cell RNA sequence studies have demonstrated little or no expression of ACE2 in vascular endothelial cells^{4,9}. These findings have left unresolved whether direct SARS-CoV-2 infection of endothelial cells contributes to coagulopathy and thrombosis.^{5,7,8}

SARS-CoV-2 enters host cells through binding of its spike protein to the ACE2 receptor.^{2,3} Like other viral spike proteins, such as those causing Ebola and Marburg hemorrhagic fever, the SARS-CoV-2 spike protein is highly glycosylated to form a “glycan shield” to evade host immune surveillance.^{10,11,12} Recently, two independent groups identified “complex type” and “high-mannose type” N-linked glycans on the SARS-CoV-2 spike protein^{10,11}, although the biological significance of these structures was unclear.

What are needed are novel compositions and methods for treating patients that have been infected with SARS-CoV-2.

### SUMMARY OF THE INVENTION

In one embodiment, the present invention includes a method for treating and/or preventing a viral or virally-induced tissue dysfunction or failure in a patient comprising administering an inhibitor of SARS-CoV-2 binding to L-SIGN in an amount effective to treat the viral or virally-induced tissue dysfunction or failure. In one aspect, the inhibitor is anti-L-SIGN antibody, a mannan, or a recombinant L-SIGN/Fc protein. In another aspect, the inhibitor is formulated in a pharmaceutical composition formulated for oral administration, direct injection, intravenous injection, infusion, local administration, for sustained release, or combinations thereof. In another aspect, the inhibitor reduces tissue damage or tissue injury by decreasing endothelial cell death in the liver or lymph node. In another aspect, the inhibitor is administered to the subject within 8, 12, 16, 24, 36, 48, 60, 72 hours, 4, 5, 6, 7, 8, 9, 10, 11, 12, 13, or 14 days from the onset of symptoms of a viral infection. In another aspect, the inhibitor is repeatedly administered to the subject at least once per day for at least 3, 4, 5, 6, 7 or more days. In another aspect, the inhibitor is provided at 20, 50, 100, 200, 500 and 1000 ug/kg/hr. In another aspect, the inhibitor is provided over 1, 2, 3, 4, 5, 6, or 7 hours via intravenous infusion. In another aspect, the inhibitor reduces damage to a tissue more than 25, 30, 33, 35, 40, 45, or 50% when compared to a non-treated tissue. In another aspect, the inhibitor reduces a vascular deficit in the liver or lymph node by at least 30, 40, 50, 60, or 70% when compared to a non-treated liver or lymph node. In another aspect, the virally-induced liver dysfunction or liver failure is a diminished microvascular blood flow in the liver caused by an immune response or a cytokine storm. In another aspect, the anti-L-SIGN antibody, a mannan, or a recombinant L-SIGN/Fc protein reduces liver tissue scarring in patients that have had a virally induced liver dysfunction or failure when compared to a non-treated liver tissue. In another aspect, the method further comprises the step of identifying the patient as having a SARS-CoV2 infection, determining if the patient is forming one or more blood clots, and providing the inhibitor within 24 hours of the earliest of: detection of the viral infection or blood clots. In another aspect, the inhibitor is injected in situ. In another aspect, cells in the viral or virally-induced tissue dysfunction or failure to not express ACE2. In another aspect, the method further comprises identifying a patient with a coronavirus infection and damaged liver or lymph node tissue;

imaging a location in a liver or lymph node of a patient with the diminished microvascular blood flow; obtaining a composition comprising a carrier solution combined with an inhibitor of Sars-CoV-2 binding to L-SIGN; and treating the liver or lymph node with an effective amount of the inhibitor.

In another embodiment, the present invention includes a method for treating and/or preventing a viral or virally-induced liver dysfunction or failure comprising administering an effective amount of an inhibitor of coronavirus binding to L-SIGN, wherein the amount of inhibitor is sufficient reduce or eliminate the viral or virally-induced liver dysfunction or failure caused by a coronavirus.

In another embodiment, the present invention includes a method for treating and/or preventing a liver or lymph node dysfunction or failure that is a virally-induced liver or lymph node dysfunction or failure, comprising administering at least one of: anti-L-SIGN antibody, a mannan, or a recombinant L-SIGN/Fc protein, wherein the viral or virally-induced condition is caused by a coronavirus. In one aspect, the coronavirus is SARS-CoV2.

In another embodiment, the present invention includes a method for treating and/or preventing a virally-induced liver or lymph node tissue dysfunction or failure, or an inflammatory condition of the liver comprising administering an effective amount of an anti-L-SIGN antibody, a mannan, or a recombinant L-SIGN/Fc protein sufficient to reduce or eliminate the damage to liver or lymph node tissue caused by a virally-induced autoimmune response to liver or lymph node tissue. In another aspect, the anti-L-SIGN antibody, a mannan, or a recombinant L-SIGN/Fc protein reduces tissue scarring in patients that have had a virally induced liver or lymph node dysfunction or failure when compared to a non-treated tissue. In another aspect, the method further comprises the step of identifying the patient as having a SARS-CoV1, MERS, or SARS-CoV2 infection, determining if the patient is forming one or more blood clots, and providing the anti-L-SIGN antibody, a mannan, or a recombinant L-SIGN/Fc protein within 24 hours of the earliest of: detection of the viral infection or blood clots. In another aspect, cells in the viral or virally-induced tissue dysfunction or failure to not express ACE2.

In another embodiment, the present invention includes a method for treating and/or preventing a viral or virally-induced formation of blood clots or epithelial cell damage in the liver or lymph node in a patient comprising: identifying the patient as having a SARS-CoV2 infection; determining if the patient is forming one or more blood clots or has or epithelial cell damage in the liver or lymph node; administering at least one of: an anti-L-SIGN antibody, a mannan, or a recombinant L-SIGN/Fc protein in an amount effective to treat the viral or virally-induced blood clot formation or epithelial cell damage, wherein the anti-L-SIGN antibody, a mannan, or a recombinant L-SIGN/Fc protein is administered within 24 hours of the earliest of: detection of the viral infection, detection of blood clots, or detection of or epithelial cell damage, wherein the anti-L-SIGN antibody, a mannan, or a recombinant L-SIGN/Fc protein prevents or reduces the effect of blood clots or epithelial cell damage in the liver or lymph node.

### BRIEF DESCRIPTION OF THE DRAWINGS

For a more complete understanding of the features and advantages of the present invention, reference is now made to the detailed description of the invention along with the accompanying figures and in which:

FIGS. 1A to 1J show Ca²⁺-dependent binding of L-SIGN to the SARS-CoV-2 spike protein mediates viral entry. FIG. 1A, Diagram of protein domain structures of SARS-CoV-2 spike protein and human L-SIGN. SS, signal sequence. TM, transmembrane domain. CP, cytoplasmic domain. EPN, Glu-Pro-Asn motif. (See also FIGS. 11 and 1J), FIG. 1B Coomassie brilliant blue stain (CBB) staining of SDS-PAGE gels of purified Spike/Fc protein under non-reducing (non-red) and reducing (red) conditions, with or without treatment of PNGaseF that removes N-glycans. FIG. 1C, Protein expression of ACE2-myc3, L-SIGN-flag, and DC-SIGN-flag in transiently transfected HEK293T cells by western blotting. GAPDH is a loading control. FIG. 1D, Representative flow cytometry plots of Spike/Fc binding to mock-transfected, ACE2-myc3-expressing, DC-SIGN-flag-expressing, and L-SIGN-flag-expressing HEK293T cells in the presence of Ca²⁺with or without EGTA at 1 mM. Dashed boxes indicate positive staining. FIG. 1E, Representative flow cytometry plots of VSVG-type (histogram) and SARS-CoV-2-type (density plot) pseudo-typed virus infection to mock-transfected, ACE2-myc3-expressing, DC-SIGN-flag-expressing, and L-SIGN-flag-expressing HEK293T cells defined by ZsGreen expression. Uninfected cells were used as negative control. Dashed boxes indicate infected cells. FIG. 1F, Quantification of pseudo-typed virus infection in (FIG. 1E). Each dot represents an individual experiment. FIG. 1G, Representative overlaid flow cytometry plots of L-SIGN expression and CoV2-type infection on L-SIGN-expressing HEK293T cells. N.C., negative control. Uninfected, L-SIGN-stained. Infected, L-SIGN-stained CoV2-type infected HEK293T cells. FIG. 1H, Infection efficiencies of CoV2-type with different types of L-SIGN internalization motif mutations (n=4). Percentages of infection were normalized with percentages of expression of L-SIGN in HEK293T cells. Bars, mean; error bars, s.e.m. *P<0.05; **P<0.01. NS, not significant. All experiments were repeated at least three times. FIGS. 1I and 1J shows the family and phylogenetic tree of L-SIGN. FIG. 1I, Alignment of primary sequences of full-length human L-SIGN and its orthologs of great apes. Highlighted areas mark di-leucine and tri-acidic cluster internalization motifs (orange) and EPN motif (blue). FIG. 1J, Phylogenetic tree of CD209 family of genes in human, non-human primates, and mouse. The phylogenetic tree was constructed by the CLUSTAL W algorithm using primary sequences of human L-SIGN and its orthologs of great apes retrieved from the Uniprot protein database. The phylogenetic tree represents evolutionary relationships based on distance among organisms. Red color marks human and great apes.

FIGS. 2A to 2K show that L-SIGN binds to a high-mannose N-glycan on the spike protein of SARS-CoV-2. FIG. 2A, Percentages of high-mannose vs complex/hybrid N-glycans in recombinant control Fc and Spike/Fc proteins determined by MALDI-TOF MS. See also FIG. 6. FIG. 2B, Left, silver staining of SDS-PAGE gel of purified Spike^{N343Q}/Fc protein or of wild-type (WT) Spike/Fc treated with or without PNGaseF. Right, western blots and GNL lectin blots of WT and Spike^{N343Q}/Fc. The reduced size of Spike^{N343Q}/Fc protein relative to that of WT indicates the loss of an N-glycan on the RBD. FIG. 2C, Representative flow cytometry plots of Spike^{N343Q}/Fc binding to mock-transfected, ACE2-myc3-expressing, and L-SIGN-flag-expressing HEK293T cells. Dashed boxes indicate positive staining. FIG. 2D, Representative flow cytometry histograms of surface expression of L-SIGN mutants, Spike/Fc binding, and VSVG-type or SARS-CoV-2-type pseudo-typed viral infections to mock-transfected HEK293T cells (mock) or transfected HEK293T cells expressing the indicated L-SIGN construct. N.C. represents either isotype-matched IgG stained, Fc control, or no viral infection. Arrows indicate positive staining. FIG. 2E, Cartoon of Spike/Fc binding and CoV2-type infection on L-SIGN expressing cells. QPD or AAA represent loss-of-function mutations of the carbohydrate recognition domain of L-SIGN. FIG. 2F, Representative flow cytometry plots of L-SIGN/Fc binding to Spike protein-expressing HEK293T cells. Mannan (1 μg/ml), a mannose polymer, was used as a competitive inhibitor of L-SIGN/Fc. L-SIGN^{QPD}/Fc is a carbohydrate recognition domain mutant of L-SIGN/Fc. EGTA (1 mM) was used as Ca2+ chelator. Dashed boxes indicate positive staining. All experiments were repeated at least three times. FIGS. 2G-2K show a schematic diagram of pseudo-type virus infection assay and internalization motif-L-SIGN mutants. FIG. 2G, Generation of lentivirus-based VSV-G and SARS-CoV-2 pseudo-type virus from HEK293T viral packaging cells. FIG. 2H, Scheme of monitoring infection based on ZsGreen expression in infected host cells. VSVG-type is pantropic virus used as positive control. FIG. 2I, Schematics of aligned internalization motif of different L-SIGN mutants. Amino acids marked with red are alanine substitution. Numbers indicate the position of amino acid in L-SIGN. FIG. 2J, Western blots of mutants of L-SIGN-flag expressed in HEK293T cells. GAPDH, loading control. FIG. 2K, Representative flow cytometry plots of L-SIGN expression (top) and CoV2-type infection (bottom) in internalization motif-mutated L-SIGN-expressing HEK293T cells. See also FIG. 1H.

FIGS. 3A and 3B show that L-SIGN, but not ACE2, is expressed on LSECs and on lymph node LECs in human biopsy or autopsy tissue samples. FIG. 3A, Representative images of immunohistochemical staining of L-SIGN (ORIGENE Cat# TA810067) in different normal human autopsy tissues. Arrows, lymphatic sinuses (left) or liver sinusoids (right). Arrowheads, blood vessels. Scale bar, 30 μm. FIG. 3B, Representative confocal microscopy images of immunofluorescent staining of different normal human autopsy liver and lymph node samples.

Arrows, LSECs (liver) or LECs (lymph node). Arrowheads, blood vessels. LSECs and LECs are VWF⁺Lyve1⁺ L-SIGN⁺. Blood vessels are VWF^{+ +} Lyve1⁻ L-SIGN⁻. DAPI, nuclear staining. Scale bar, 20 μm. The results represent at least three experiments from biopsy or autopsy samples of three uninfected patients.

FIGS. 4A to 4E shows that LSECs of COVID-19 patient liver autopsy samples have detectable SARS-CoV-2 and express increased levels of VWF and FVIII. FIG. 4A, Representative confocal microscopy images of SARS-CoV-2 nucleocapsid protein in LSECs of a COVID-19 liver autopsy sample. Uninfected normal human liver autopsy sample was used as a negative control. LSECs are L-SIGN positive (green). Arrows mark SARS-CoV-2 (red). DAPI (grey), nuclear staining. Scale bar, 10 μm. FIG. 4B, Representative 3-D rendering of Z-stacked confocal microscopy images of liver sections from normal human and COVID-19 liver autopsy samples. Scale bars, 20 μm. FIG. 4C, Representative single-color or merged-color confocal images of immune-stained human liver sections. Arrow, central vein. L-SIGN marks LSECs. DAPI (white), nuclear staining. Scale bar, 20 μm. FIG. 4D, Quantification of relative mean fluorescence intensity of VWF or FVIII associated with L-SIGN-positive LSECs. FIG. 4E,

Representative confocal microscopy images of Lyve1 (red) in a COVID-19 liver autopsy sample. Uninfected normal human liver autopsy sample was used as a positive control. Scale bar, 10 μm. **P<0.01; NS, not significant. Bars, mean; error bars, s.e.m. The results represent data from biopsy of autopsy samples of three independent COVID-19 patients and three uninfected patients.

FIGS. 5A to 5B show that blocking L-SIGN function reduces CoV2-type infection. FIG. 5A, Schematic diagram depicts inhibitory mechanisms of L-SIGN-mediated CoV2-type infection by mannan, L-SIGN/Fc, and anti-L-SIGN. FIG. 5B, Inhibition assay of L-SIGN-mediated CoV2-type infection using mouse anti-human L-SIGN (10 and 2 μg/m1), mannan (500 and 100 μg/m1), or recombinant L-SIGN/Fc (50 and 10 μg/m1 corresponding to 1 and 0.2 nanomole/ml). Isotype-matched control IgG (10 and 2 μg/m1) and Fc control (1 and 0.2 nanomole/ml) were used as negative control. % Infection efficiency for each inhibiter was normalized against untreated CoV2-infected L-SIGN-expressing cells. Negative control is isotype-matched-IgG-stained, mock-transduced cells. See also FIGS. 7E, 7F. *P<0.0001. Bars, mean; error bars, s.e.m. All experiments were repeated three times.

FIGS. 6A to 6D show that Spike/Fc has high-mannose type N-glycanS. FIG. 6A, FIG. 6B. Glycan structure analyses of control Fc and Spike/Fc by MALDI-TOF mass spectrometry. The proposed structures of the glycans are shown with their corresponding ion peak in the MALDI-TOF MS spectrum. Arrows indicate major high-mannose type N-glycan structures. FIG. 6C, CBB staining of SDS-PAGE gels of purified Spike/Fc protein under reducing conditions, with or without PNGaseF or Endo-H treatment. FIG. 6D, Primary sequence of receptor binding domain surrounding an N-glycosylation site (N343) of SARS-CoV-2 spike protein, which is conserved in receptor binding domain of SARS-CoV as marked with red. Underline is consensus N-glycosylation site (Asn-X-Ser/Thr). X cannot be Pro.

FIGS. 7A to 7F show that L-SIGN recognizes high-mannose type N-glycans on SARS-CoV-2 spike protein. FIG. 7A, CBB-stained SDS-PAGE gel of purified L-SIGN/Fc and L-SIGN^{QPD}/Fc proteins. FIG. 7B, Schematic diagram of N-glycan biosynthetic pathway. Kifunensine is an inhibitor of mannosidase I, resulting in accumulation of high-mannose type N-glycans. FIG. 7C, Representative histograms of flow cytometry of L-SIGN/Fc binding and biotin-labeled GNL binding to spike-protein-expressing HEK293T cells in the presence of kifunensine (10 μM, 48 h). Treatment with DMSO was used as negative control. N.C. is mock transfected cells stained with Fc control or PE-streptavidin only. FIG. 7D, Cartoon of glycans on cell surface of kifunensine-treated spike-protein-expressing HEK293T cells. FIG. 7E Mechanisms of VSVG-type pantropic viral infection. LDLR, low density lipoprotein receptor. FIG. 7F, Inhibition assay of VSVG-type infection in HEK293T cells. % Infection efficiency for each inhibiter was normalized against untreated VSVG-infected HEK293T cells. See also FIG. 5A, 5B.

FIG. 8 shows the L-SIGN expression profile in different human tissues. Images of immunohistochemical stained formalin-fixed paraffin-embedded sections of human autopsy tissue samples. Sections were incubated with mouse anti-human L-SIGN mAb (Origene Cat# TA810067, 1:150). Negative control, isotype-matched control IgG. Scale bar, 25 μm.

FIG. 9 shows the validation of L-SIGN expression profile with a different anti-L-SIGN antibody. Representative confocal images of immunofluorescent stained formalin-fixed paraffin-embedded sections of human autopsy tissue samples. Lyve1 (green) is LSECs (liver) or LECs (organs other than liver). Scale bar, 10 μm. Sections were incubated with mouse anti-human L-SIGN monoclonal Ab (ORIGENE Cat# TA810055).

FIGS. 10A and 10B show that ACE2 is not expressed in LSECs or in lymph node LECs. FIG. 10A, Representative confocal images of immunofluorescent stained formalin-fixed paraffin-embedded sections of human autopsy tissue samples. Lyve1 (red) positive cells are LSECs (liver) and LECs (lymph node), DAPI is nuclear counter staining. Kidney is positive control for ACE2 staining (green). Scale bar, 10 μm. FIG. 10B, Co-expression profiles of genes relevant to this study in L-SIGN-positive LSECs, which was extracted from a published database of single cell RNA transcriptional profiles of 8,444 cells from five human liver samples. Similar transcriptome populations were grouped by color-assigned clusters. LYVE1 and STAB1, LSEC-specific genes. ALB (hepatocyte specific), CD3E (T lymphocyte specific), and CD68 (Kupffer cell-specific), non-LSEC-specific genes. Red circles indicate LSEC clusters. L-SIGN and coagulation-related genes as highlighted by bigger blue dots are enriched in LSEC clusters.

FIGS. 11A to 11G show that cultured vascular endothelial cells do not express L-SIGN. FIG. 11A, Representative flow cytometry histogram showing specificity of anti-L-SIGN mAb. Mock-, L-SIGN- or DC-SIGN-transiently expressing HEK293T cells were stained with mouse anti-human L-SIGN mAb. Isotype-matched control IgG and mock transfected cells were negative controls. FIG. 11B, Representative overlaid flow cytometry histogram of L-SIGN expression in three different human endothelial cell lines (EAhy926, immortalized HUVECs, and immortalized DMECs) without or with stimulation with TNFα (10 ng/ml) or IL-4 (10 ng/ml) for 24 h. FIG. 11C, Representative flow cytometry histogram of ACE2 expression in human endothelial cells. HEK293T cells expressing ACE2-myc3 were positive control. FIG. 11D, Representative flow cytometry histogram of VSVG-type and CoV2-type infection of human endothelial cells. FIG. 11E, Representative overlaid dot plots of flow cytometry of L-SIGN expression in primary liver sinusoid endothelial cells and lymphatic endothelial cells. Cells were co-stained with anti-CD31 antibody. Red, isotype-matched IgG-stained. Blue, antibody-stained cells. HEK293T cells expressing L-SIGN-flag were positive control for L-SIGN staining. FIG. 11F, Representative overlaid flow cytometry histogram of L-SIGN expression in LSECs stimulated with TNFα (10 ng/ml), IL-6 (10 ng/ml) or combination for 24 h. All experiments were repeated at least three times. FIG. 11G, Representative overlaid flow cytometry plots of L-SIGN expression and CoV2-type infection on L-SIGN-transduced LSECs cells. N.C., negative control. Uninfected, L-SIGN-stained. Infected, L-SIGN-stained CoV2-type infected LSECs.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “treating,” “treatment,” “therapeutic,” or “therapy” do not necessarily mean total cure or abolition of the disease or condition, but rather, include any alleviation of any undesired signs or symptoms of a disease or condition, to any extent, can be considered treatment and/or therapy. It is entirely possible that “treatment” includes a temporary improvement of tissue damage or tissue injury by decreasing endothelial cell death in the liver or lymph node that requires repeated treatment over time to continue the regenerative process. Furthermore, treatment may include acts that may worsen the patient's overall feeling of well-being or appearance.

As used herein, the phrase “therapeutically effective amount” refers to a compound as used herein to indicate an amount of an active compound, or pharmaceutical agent, that elicits the biological or medicinal response indicated, e.g., anti-L-SIGN antibody, a mannan, or a recombinant L-SIGN/Fc protein. This response may occur in a tissue, system, animal or human and includes alleviation of the symptoms of the disease being treated. The exact formulation, route of administration and dosage for the composition and pharmaceutical compositions disclosed herein can be chosen by the individual physician in view of the patient's condition. (See e.g., Fingl et al. 1975, in “The Pharmacological Basis of Therapeutics”, Chapter 1, and updates thereof, or Remington's Pharmaceutical Sciences, Mace Publishing Company, Philadelphia, Pa., 17th ed. (1985) and updates thereof, relevant portions incorporated herein by reference). Therapeutic treatments can be achieved with small molecule organic drugs or biologics, such as proteins. Typically, the dose range of the anti-L-SIGN antibody, a mannan, or a recombinant L-SIGN/Fc protein therapeutic agent is administered from about 0.5 to 1000 μg/kg, or 1 to 500 μg/kg, or 10 to 500 μg/kg, or 50 to 100 μg/kg of the patient's body weight per dose. The dose of a therapeutic anti-L-SIGN antibody, a mannan, or a recombinant L-SIGN/Fc protein can be administered to the patient intravenously or intraarterially as either a bolus dose or by infusion from about 0.1 to 100 μg/kg of the patient's body weight, or 0.3 to 30 μg/kg, or 1 to 3 μg/kg of the patient's body weight per dose. To achieve localized targeted dosing, anti-L-SIGN antibody, a mannan, or a recombinant L-SIGN/Fc protein can be injected either directly into or adjacent to the tissue damage or tissue injury by decreasing endothelial cell death in the liver or lymph node, preferably either into or as near as practical to the region of viral damage. Localized dose ranges can be from 10 ng/cm³ to 1 mg/cm³, or 100 ug/cm³ to 100 ug/cm³ or 1 ug/cm³ to 10 ug/cm³ of target tissue damage or tissue injury by decreasing endothelial cell death in the liver or lymph node per dose. Local doses can be administered the liver or lymph node(s), or upstream thereof, in the case of the liver intravenously and in the case of lymph nodes into one or more lymphatic drainage area(s). The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

In some embodiments a carrier solution or containing/metering device may be desired to deliver the anti-L-SIGN antibody, a mannan, or a recombinant L-SIGN/Fc protein. Appropriate carrier solutions may be selected based on properties such as viscosity, ease of administration, ability to bind solution over a period of time, and general affinity for the agent delivered. Such solutions may be modified or additives incorporated for modification of biological properties.

As used herein, the term “subject” refers to any chordates, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “effective dose” refers to that amount of anti-L-SIGN antibody, a mannan, or a recombinant L-SIGN/Fc protein to prevent and/or ameliorate an infection or to reduce at least one symptom of an infection and/or to enhance the efficacy of another dose of a coronavirus. An effective dose may refer to the amount of the anti-L-SIGN antibody, a mannan, or a recombinant L-SIGN/Fc protein sufficient to delay or minimize the onset of an infection. An effective dose may also refer to the anti-L-SIGN antibody, a mannan, or a recombinant L-SIGN/Fc protein in an amount that provides a therapeutic benefit in the treatment or management of an infection. Further, an effective dose is the amount with respect to the anti-L-SIGN antibody, a mannan, or a recombinant L-SIGN/Fc protein of the invention alone, or in combination with other therapies, that provides a therapeutic benefit in the treatment or management of an infection. An “effective dose” of the anti-L-SIGN antibody, a mannan, or a recombinant L-SIGN/Fc protein is one that prevents disease and/or reduces the severity of symptoms.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

The present inventors determined that SARS-CoV-2 is related to SARS-CoV (˜80% sequence identity), which was the coronavirus that caused the epidemic of severe acute respiratory syndrome in 2003. Previous studies have shown that SARS-CoV, in addition to interact with ACE2, binds to liver/lymph node-specific ICAM-3 grabbing non-integrin (L-SIGN, also known as CD209L, CD299 or CLEC4M), which mediates virus entry into host cells. L-SIGN is a C-type lectin receptor that mediates cell adhesion or pathogen recognition by Ca²⁺-dependent binding to glycans. Whether SARS-CoV-2 uses L-SIGN to infect host cells is unknown. The inventors found that L-SIGN bound to SARS-CoV-2 spike protein in a high-mannose N-glycan- and Ca²⁺-dependent manner. L-SIGN was detected at high levels on liver sinusoidal endothelial cells (LSECs) and on lymphatic endothelial cells (LECs) of lymph nodes from human biopsy or autopsy samples. LSECs and LECs are major sources of procoagulant proteins such as FVIII and VWF. The inventors also found that increased expression of VWF and FVIII in LSECs of COVID-19 patient liver samples. This study reveals a SARS-CoV-2 receptor on endothelial cells contributes to COVID-19-associated coagulopathy.

Materials and methods. Plasmids. Plasmids of human ACE2, SARS-CoV2-Spike, pLVX-M-puro were purchased from Addgene (Cat# 1786, 145032, 125839). The full cDNA sequence of the human ACE2 was subcloned into pcDNA3.1(+)/myc3 to express C-terminal myc3-tagged human ACE2. Plasmids of C-terminal flag-tagged L-SIGN, DC-SIGN, and TMPRSS2 were purchased from Genscript (Cat# OHu19035, OHu23087, OHu13675). The cDNA sequence of the extracellular domain of human L-SIGN (residues 78-399) was sub-cloned into pFUSEN-hG2Fc plasmid (InvivoGen Cat# pfcn-hg2) to express N-terminal human Fc-tagged human L-SIGN (L-SIGN/Fc) recombinant soluble protein. The cDNA sequence of the SARS-CoV2 Spike RBD (residues 333-529) was sub-cloned from pcDNA3-SARS-CoV-2-S-RBD-Fc (Addgene Cat# 141183) into pFUSE-hIgG2-Fc2 plasmid (InvivoGen Cat# pfuse-hfc2) to express C-terminal human Fc-tagged Spike RBD (Spike/Fc) recombinant soluble protein. The cDNA sequence of the L-SIGN was sub-cloned into pLVX-M-puro. N343Q-carrying Spike/Fc mutant expression vectors, mutated EPN motif-carrying L-SIGN expression vectors, and mutated endocytosis motif-carrying L-SIGN expression vectors were generated by inverse PCR using KOD Xtreme DNA polymerase (Millipore Sigma Cat# 71975). pHIV-ZsGreen plasmid was a kind gift from Dr. Bryan E. Welm (University of Utah). Plasmids of psPAX2 and pMD2.G were purchased from Addgene (Cat# 12260, 12259).

Protein purification. COS-7 cells in 20×100 mm dishes transfected with plasmid by PEI-max (MW 4,000) (Polysciences, Inc. Cat# 24885) were cultured for 3 days in serum-free Opti-MEM1 media (Invitrogen Cat# 51985034) containing ITS-X (Gibco Cat# 51500056), glutamine/penicillin/streptomycin, non-essential amino acids, sodium pyruvate, and 0.5% yeast extract (BD Biosciences Cat# 212750). Culture medium was centrifuged at 3,000 g for 10 min, and supernatant was filtered through a 0.22 ₈₂ m membrane. After adjusting pH with Tris-HCl buffer to pH 7.6, secreted L-SIGN/Fc, Spike/Fc, and Fc control were purified from the supernatant using Protein A agarose (Goldbio Cat# P-400-5). Afterward, the buffer was exchanged to PBS using a 30 K cut-off amicon filter tube (Millipore Corp Cat# UFC803008), protein concentration was measured by DC protein assay (Bio-Rad Cat# 5000111). Purity of proteins was examined by staining gel and membrane with Coomassie Brilliant Blue and Ponceau S solution, respectively. Silver staining was carried out according to manufacturer's protocol (Thermo Fisher Cat# 24612).

Cell culture. COS-7 cells and HEK293T cells purchased from ATCC (Cat# CRL-1651, CRL-3216) were grown in DMEM (Corning # 10-013-CV) supplemented with 10% FBS, 100 U/ml penicillin, and 100 μg/ml streptomycin. Human dermal microvascular endothelial cells and human liver-derived sinusoid endothelial cells purchased from Lonza (Cat# CC-2543, HLECP2) were grown in EGM-2 medium (Lonza Cat# CC-3162).

Reagents. Anti-human L-SIGN mouse monoclonal antibody (mAb) for western blotting, flow cytometry, and infection inhibition assay, and anti-human ACE2 antibody for flow cytometry were purchased from R&D Systems (Cat# MAB162, AF933). Anti-human ACE2 mouse mAb for tissue immunohistostaining was purchased from Abcam (Cat# ab89111). Anti-human L-SIGN Abs for tissue immunohistostaining were purchased from ORIGENE (Cat# TA810067, TA810055 and Sigma Cat# AV42396). Anti-flag and anti-myc antibodies were obtained from Santa Cruz Biotechnology (Cat# sc-166355, sc-40). Anti-coagulation factor VIII antibody was purchased from LSBio (Cat# LS-B2979). Anti-SARS nucleocapsid protein antibodies were from Novus Biologicals (Cat# NB100-56576). PNGaseF, Endo-H and α1-2,3,6 mannosidase were purchased from NEB (Cat# P0704S, P0702S, P0768S). Neuraminidase was from Millipore Sigma (Cat# 10269611001). Mannan was purchased from Sigma (Cat# M7504). Kifunensine was purchased from Cayman Chemical (Cat# 10009437).

SARS-CoV-2 pseudovirus production and infection inhibition assay. HEK293T cells (1×10⁶ cells) were plated on 6-well plates. On the following day, pHIV-ZsGreen (3.3 μg), psPAX2 (2.5 μg) and SARS-CoV2-Spike (1 μg) were co-transfected, and after 15 h, medium was replaced with fresh medium. On the following day, culture medium containing virus was aseptically centrifuged at 15,000 g for 10 min at room temperature, and after adding polybrene (2 μg/ml), SARS-CoV-2 pseudovirus-containing medium was added to target cells. After 48 h, infection efficiency, defined by ZsGreen expression, was determined by flow cytometry. VSVG-type pseudovirus was generated by co-transfecting of pHIV-ZsGreen (3.3 μg), psPAX2 (2.5 μg), and pMD2.G (1.67 μg) as positive control. For the infection inhibition assay, mouse anti-human L-SIGN mAb (10 and 2 μg/ml) or mannan (500 and 100 μg/ml) were added at the same timing of the infection. The L-SIGN/Fc recombinant protein (50 and 10 μg/ml corresponding to 1 and 0.2 nanomole/ml) were pre-incubated with the virus-containing media for 15 min at room temperature before initiating the infection. Isotype-matched control IgG (10 and 2 μg/ml) and Fc control (1 and 0.2 nanomole/ml) were used as negative controls.

Flow cytometric analysis. To examine Spike/Fc binding to cells, plasmids coding for human ACE2, L-SIGN, and DC-SIGN were transfected in HEK293T cells, and after 48 h, cells were incubated with Spike/Fc (50 μg/m1) in Hanks' balanced salt solution (HBSS) (Corning-Cat# 21-023-CV) on ice for 1 h. After washing with HBSS, cells were further stained with Alexa Fluor 488-conjugated anti-human IgG (Jackson ImmunoResearch Cat# 709-545-098) on ice for 30 min. After washing again, cells were analyzed on FACSCelesta, and data were analyzed using FlowJo software. To examine L-SIGN/Fc binding to cells, the plasmid coding for Spike was transfected in HEK293T cells, and after 48 h, cells were incubated with L-SIGN/Fc (1 μg/ml) on ice for 1 h. Control Fc was used as a negative control. For the inhibition assay, L-SIGN/Fc was pre-incubated with mannan (1 μg/ml) for 15 min at room temperature. L-SIGN expression was examined using anti-L-SIGN specific mAb (R&D Systems Cat# MAB162). For lectin staining, cells were incubated with GNL (2 μg/ml) on ice for 1 h. After washing with HBSS, cells were further stained with PE-conjugated streptavidin (Jackson ImmunoResearch Cat# 016-110-084) on ice for 30 min. Double staining of CD31 and L-SIGN were done by staining cells with rabbit anti-human CD31 pAb (Santa Cruz Biotechnology Cat# sc-8303) and anti-L-SIGN mAb followed by secondary staining with fluorescence-conjugated antibodies. The infection efficiency among different endocytosis mutants was normalized based on the %surface expression of L-SIGN.

Western blotting and lectin blotting. Protein extracts from cultured cells or tissues were prepared using a 1% Triton X-100-based cell lysis buffer (Cell Signaling Technology Cat# 9803). Lysates were applied to an 8-12% SDS-PAGE gel and separated proteins were transferred onto a PVDF membrane (Thermo Fisher Cat# 88518). After blots were blocked with skim milk in TBS-0.05% Tween20 (TBS-T), the membranes were incubated with primary and HRP-conjugated secondary antibodies and developed with ECL systems (Thermo Fisher Cat# 34577). For the GNL lectin staining, transferred PVDF membranes were blocked with 5% BSA in TBS-T overnight at 4° C., and incubated with biotin-conjugated GNL (1 μg/ml) in 5% BSA in TBS-T for 1 h at room temperature. After washing, the membrane was incubated with HRP-conjugated streptavidin (Jackson ImmunoResearch Cat# 016-030-084) in 5% BSA in TBS-T for 30 min at room temperature. Exposed X-ray films (MIDSCI Cat# BX57) were scanned and analyzed with ImageJ software (NIH).

Histology and microscopy. De-identified formalin-fixed paraffin-embedded biopsy or autopsy tissues from three uninfected patients as well as de-identified formalin-fixed paraffin-embedded autopsy samples of three COVID-19 patients were used.

For histology, paraffin sections (5 μm) were stained with hematoxylin and eosin. For immunohistochemistry, paraffin sections were deparaffinized and rehydrated. For antigen retrieval, heat-induced antigen retrieval was performed in 1 mM EDTA in 10 mM Tris buffer (pH 8.5) at 120° C. in an autoclave for 3 min. In some experiments, paraffin sections were deparaffinized and incubated with 6 M guanidine hydrochloride for 30 min at room temperature.

After wash, sections were then treated with 0.2 mg/ml trypsin for 30 min at 37° C. Endogenous peroxidase was inactivated by incubation with 3% hydrogen peroxide for 15 min at room temperature. For staining, sections were incubated with mouse anti-human L-SIGN mAb (Origene Cat# TA810067, TA810055) or rabbit anti-human L-SIGN polyclonal Ab (Sigma Cat# AV42396, 1:100) for 1.5 h at room temperature. Secondary antibodies (5 μg/ml, HRP-conjugated goat anti-mouse IgG or HRP-conjugated goat anti-rabbit IgG) were added for 15 min at room temperature. The staining was visualized using diaminobenzidine substrate (Vector Laboratories Cat# SK-4100).

For immunofluorescent staining, paraffin sections were deparaffinized and rehydrated. Heat-induced antigen retrieval was performed by citrate buffer (pH 6.0) at 100° C. for 20 min or by 1 mM EDTA in 10 mM Tris buffer (pH 8.5) at 120° C. for 3 min. Paraffin sections were blocked for 3 h at room temperature and incubated with primary antibodies overnight at 4 ° C. Secondary antibodies were added for 45 min at room temperature. Primary antibodies were to CD31 (Dako Cat# M0823, 1:20), VWF (Dako Cat# A0082, 1:200), SARS nucleocapsid protein (Novus Biologicals Cat# NB100-56576, 1:50), Lyve1 (Abcam Cat# ab219556, 1:3000), ACE2 (Abcam Cat# ab89111, R&D Systems Cat# AF933, 1:200), L-SIGN (Origene Cat# TA810067 and TA810055, 1:150), Factor VIII (LSBio Cat# LS-B2979, 1:200). Secondary antibodies were to Cy3-conjugated donkey anti-sheep IgG (Jackson ImmunoResearch Cat# 713-165-003), Cy3-conjugated donkey anti-mouse IgG (Jackson ImmunoResearch Cat# 715-165-150), Alexa Fluor 488-conjugated goat anti-mouse IgG (Jackson ImmunoResearch Cat# 115-546-062), Alexa Fluor 647-conjugated donkey anti-mouse IgG (Abcam Cat# ab150107), Alexa Fluor 488-conjugated goat anti-rabbit IgG (Jackson ImmunoResearch Cat# 111-546-003), Alexa Fluor 555-conjugated donkey anti-rabbit IgG (Abcam Cat# ab150074). The secondary antibodies were used at a concentration of 8 μg/ml.

A confocal microscope (Zeiss 710 Microscope System) was used for imaging. Z-stack images were collected at 2-μm steps with sequential laser excitation to eliminate bleed-through and with confocal parameters selected to minimize the thickness of the calculated optical section using the Zeiss software ZEN. Volume images from the confocal image datasets were further processed with IMARIS software (Bitplane AG), and some images were presented as maximum intensity projections of the z-stacks. Mean fluorescent intensity measurements of VWF and FVIII were determined from independent 16-bit tiff images. The co-stained L-SIGN was used to outline the liver sinusoidal endothelial cells with the tracer tool in ImageJ. At least 4 40× images per patient were analyzed for VWF or FVIII fluorescent intensity and the values were averaged. Tukey's test was used when comparing between groups.

Glycan structural analysis. Spike/Fc protein (0.15 mg) in PBS was sequentially mixed with 4 volumes of methanol, 1 volume of chloroform, and 3 volumes of water. After spinning at 15,000 g for 1 min, supernatant was discarded, and 4 volumes of methanol was added. After spinning at 15,000 g for 2 min, all solvent was discarded and samples were dried and dissolved in Ambic buffer. The solution was incubated with porcine trypsin (1:50) at 37° C. overnight and then heated at 100° C. for 5 min. The peptides and glycopeptides were isolated by passing through a C18 spin column, and eluted by addition of 80% acetonitrile, before drying by a SpeedVac. The dried glycopeptide eluate was treated with PNGase F to release the N-linked glycans by digestion overnight at 37° C. The digested sample was then passed through a C18 sep pak cartridge to recover the N-glycans in the 5% acetic acid flow through. This fraction was then dried by lyophilization. The dried N-glycans were then permethylated for structural characterization by mass spectrometry. Briefly, the dried eluate was dissolved in dimethyl sulfoxide and methylated with NaOH and methyl iodide. The reaction was quenched with water and per-O-methylated carbohydrates were extracted with methylene chloride and dried under N2. The permethylated samples were dissolved in methanol and crystallized with α-dihydroxybenzoic acid (DHBA) matrix (20 mg/mL in 50% methanol/water). One μ1 of each sample and the matrix were combined on the plate and allowed to dry. Analysis was performed by MALDI-TOF-MS using AB SCIEX TOF/TOF 5800 (Applied Biosystem MDS Analytical Technologies). Data were collected from a mass ranging from 800 to 4,500 daltons. The focus mass was set for 2,500 for N-glycans. Following acquisition, the baseline was smoothed and the detection limits for peaks were set to 3 SN.

Statistics. Statistical tests were performed using Prism software (GraphPad). Two-sided, two-tailed Student's t tests were performed to assess the statistical significance of differences between 2 groups after the data were confirmed to fulfill the criteria of normal distribution and equal variance. One-way analysis of variance was used to analyze the significance of differences among 3 or more groups. Differences were considered statistically significant at P<0.05. No data points was excluded from the analysis performed in this study.

Study approval. The patient study was approved by the Institutional Review Board of the Oklahoma Medical Research Foundation.

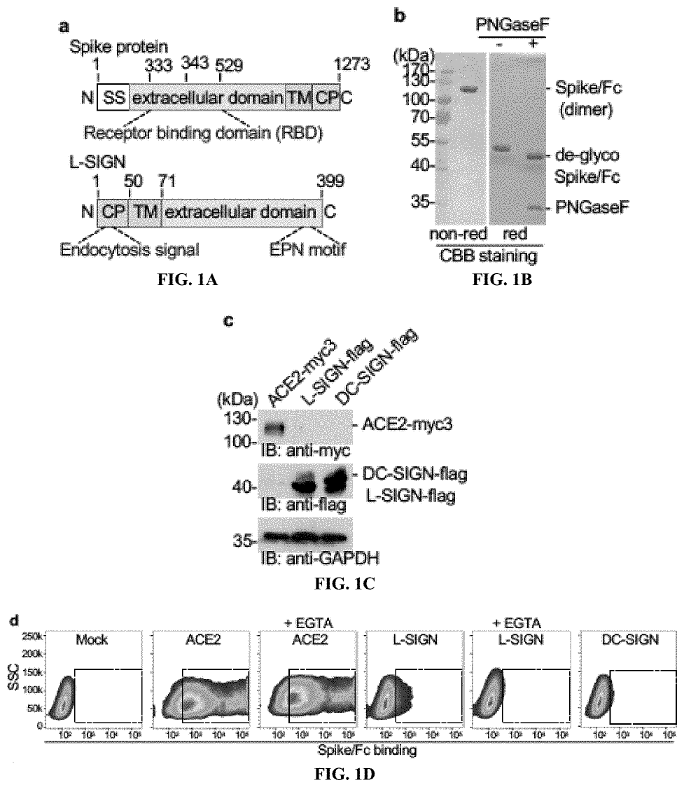

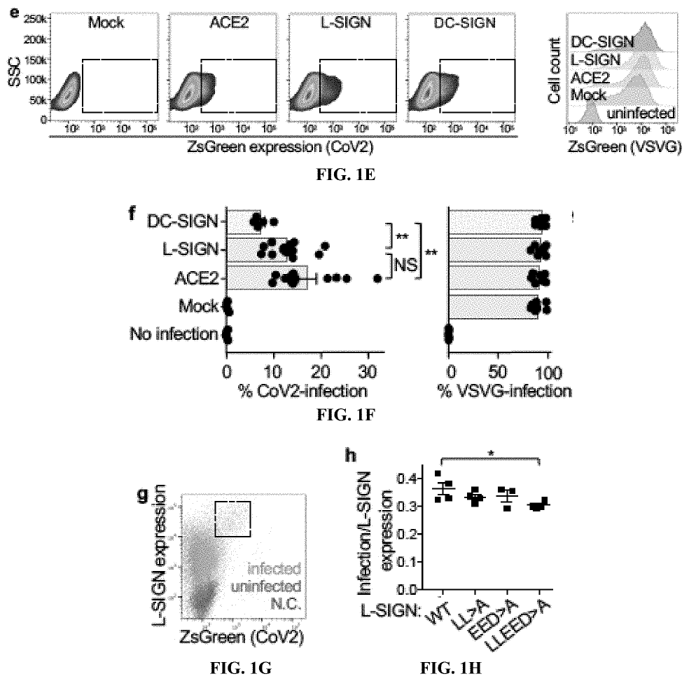

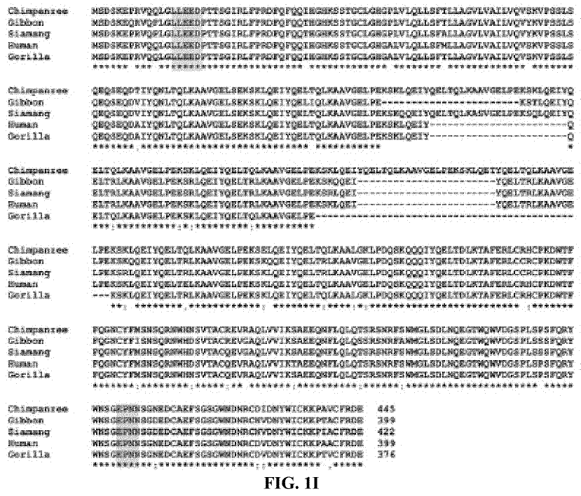

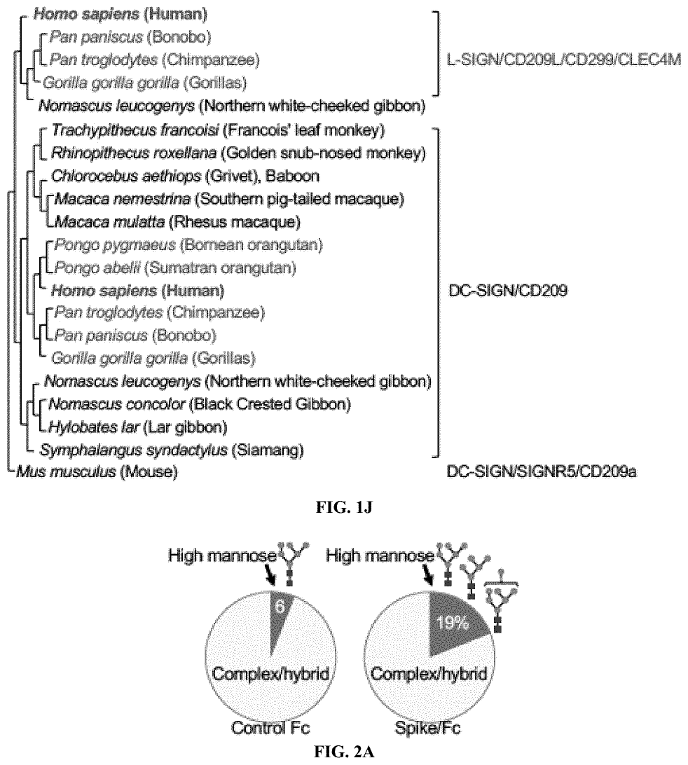

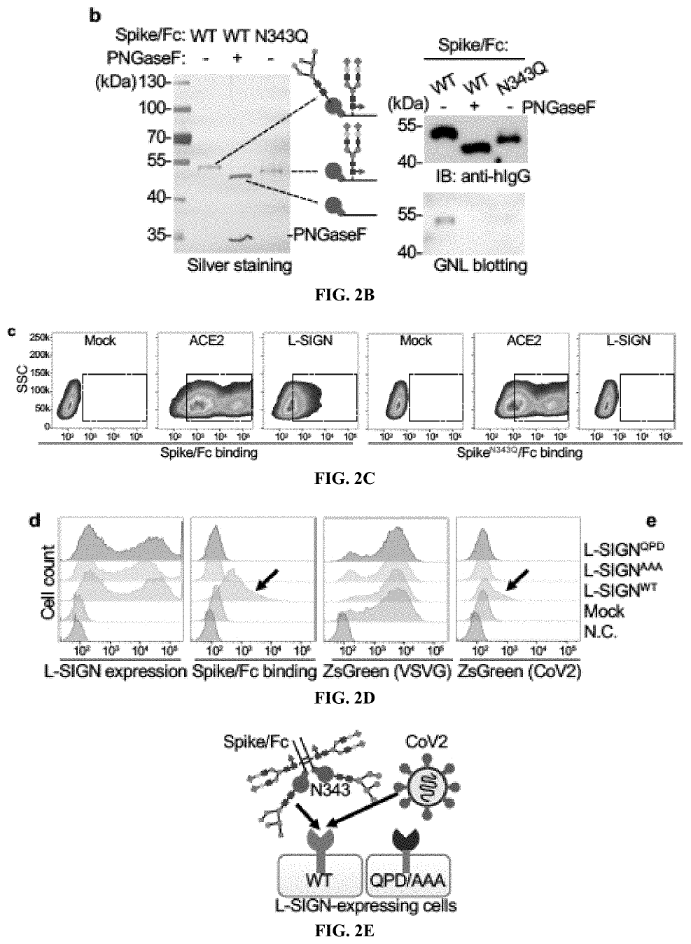

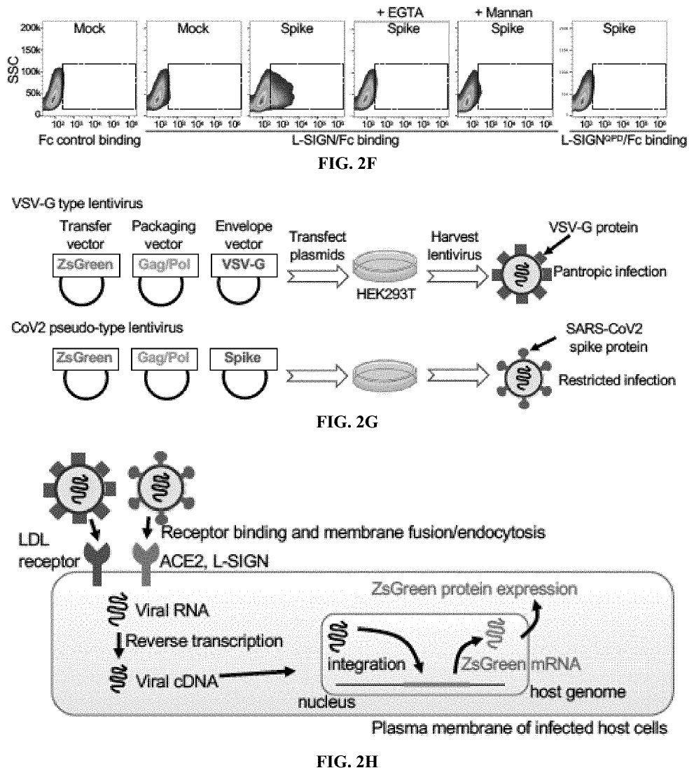

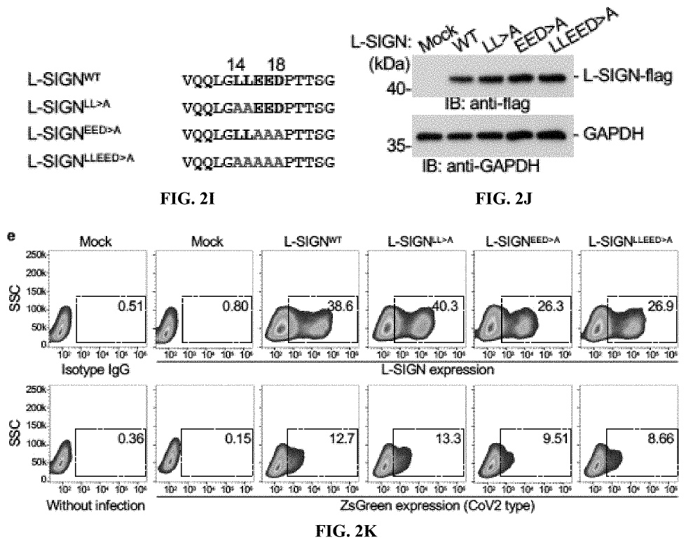

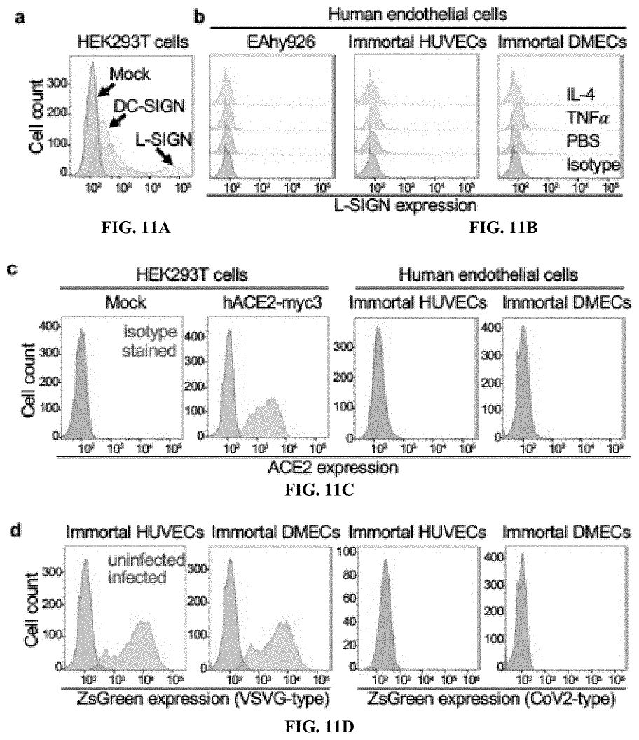

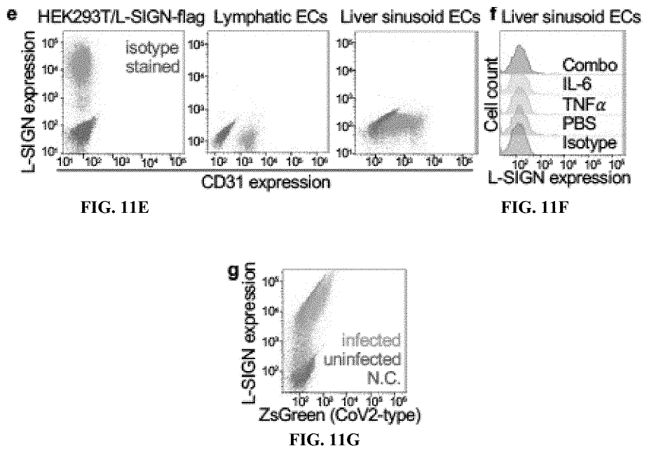

Ca²⁺-dependent binding of L-SIGN to the SARS-CoV-2 spike protein mediates viral entry. The extracellular domain of the SARS-CoV-2 spike protein has a receptor binding domain (RBD) that is modified by N-linked glycans (FIG. 1A)^{10, 11}. L-SIGN has a Glu-Pro-Asn (EPN) motif in its extracellular domain that recognizes mannose moieties (FIG. 1A, FIG. 11)^{19, 20}. The inventors generated a recombinant protein with the RBD (residue 333-529 amino acids) of the spike protein fused to a human IgG2 Fc (Spike/Fc, FIG. 1B). The inventors also established HEK293T cell lines expressing L-SIGN-flag or ACE2-myc3 as positive controls (FIG. 1C)². Spike/Fc bound to L-SIGN- and ACE2-expressing cells in Hank's balanced salt solution (HBSS) containing Ca²⁺(FIG. 1D). The Ca²⁺ chelator EGTA blocked the interaction of spike/Fc with L-SIGN but not with ACE2²⁰, indicating that binding to L-SIGN is Ca²⁺ dependent. To validate that L-SIGN is a functional receptor for SARS-CoV-2, the inventors generated SARS-CoV-2-type pseudo-typed virus (CoV2-type) (FIG. 2G, 2H)². The inventors found that CoV2-type specifically infected cells expressing ACE2 or L-SIGN (FIG. 1E, 1F). In addition, CoV2-type preferentially infected cells with higher levels of L-SIGN expression (FIG. 1G). In comparison, the control VSVG-type pseudo-typed virus (VSVG-type) non-specifically infected HEK293T cells (FIG. 1E, 1F).

L-SIGN has over 70% homology with human dendritic cell-specific CD209 (DC-SIGN), which is also expressed on monocytes and alveolar macrophages (FIG. 1J). However, Spike/Fc binding to and CoV2-type infection of DC-SIGN-expressing cells were much lower than for L-SIGN-expressing cells (FIG. 1C-1F).

L-SIGN has two distinct internalization motifs in its cytosolic domain. One is a di-leucine and the other is a tri-acidic cluster²¹. Mutagenesis of the di-leucine or the tri-acidic cluster to alanine did not affect efficiency of CoV2-type infection; however, combined mutagenesis of these two motifs significantly decreased CoV2-type infection, supporting a role of L-SIGN as an entry receptor for SARS-CoV-2 infection (FIG. 1H, FIG. 2I-K).

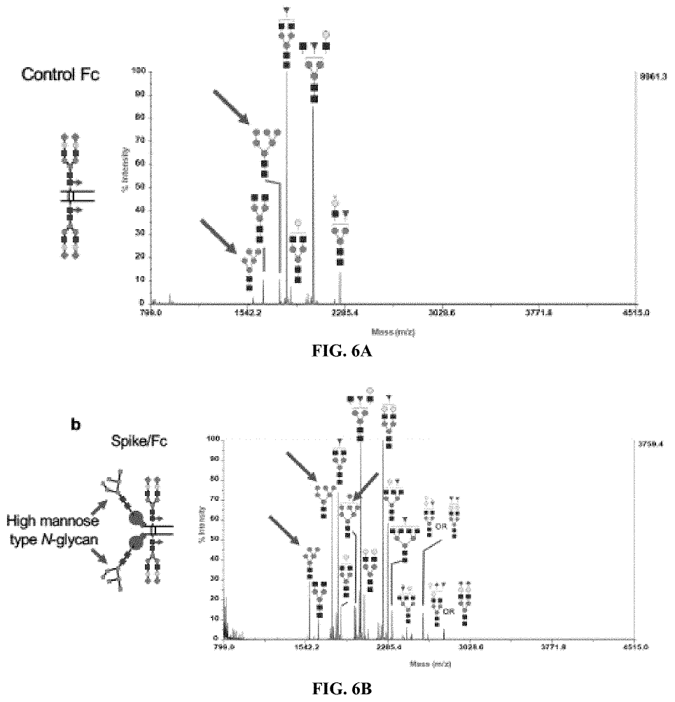

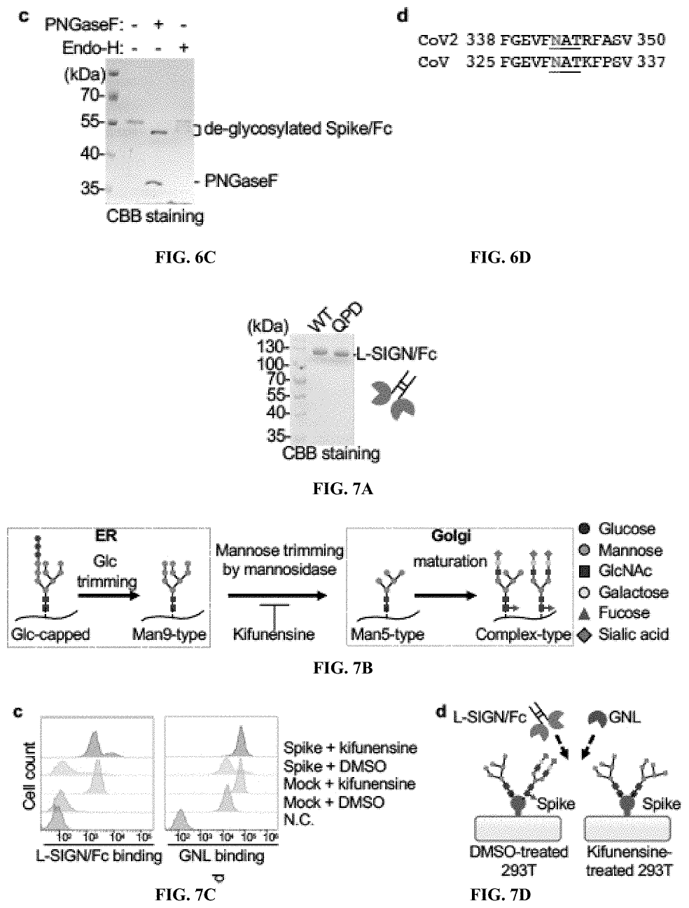

L-SIGN binds to a high-mannose N-glycan on the spike protein of SARS-CoV-2. The RBD sequence of the SARS-CoV-2 spike protein has an N-glycosylation site at Asn343^{10, 11} with unknown function (FIG. 1A). The inventors analyzed the glycan structures of Spike/Fc and found that high-mannose type N-glycans were frequent (FIG. 2A, FIG. 6A, 6B)²², which was confirmed by their removal with the high-mannose-specific endoglycosidase H (FIG. 6C). To determine whether a high-mannose N-glycan is the determinant of L-SIGN binding, the inventors first generated an N-glycan-deficient Spike/Fc in which Asn343 was replaced with glutamine (Q) (Spike^{N343Q}/Fc, FIG. 2B, left). Mannose moieties on Spike/Fc and their deficiency on Spike^{N343Q}/Fc were demonstrated by mannose-specific lectin GNL blotting (FIG. 2B, right). Spike^{N343Q}/Fc bound to cells expressing ACE2 but not to cells expressing L-SIGN, indicating that L-SIGN recognizes an N-glycan structure on N343 (FIG. 2C). To examine if the EPN motif of L-SIGN is involved in N-glycan recognition on Spike/Fc, the inventors substituted the EPN sequence with alanines (L-SIGN^{AAA}) or with Gln-Pro-Asp (QPD), a galactose recognition motif (L-SIGN^{QPD})²³. Spike/Fc binding to and CoV2-type infection of cells expressing either L-SIGN mutant were abolished (FIG. 2D, 2E).

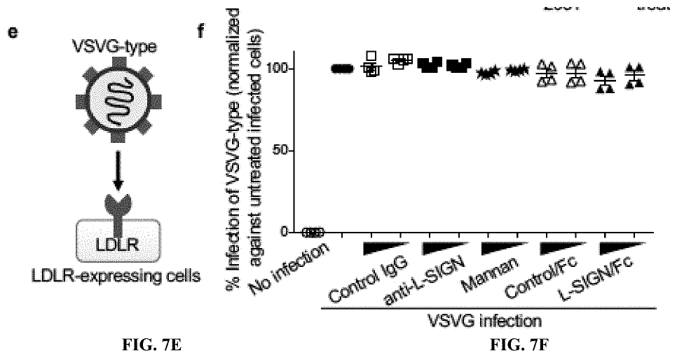

To further investigate if a high-mannose N-glycan on Spike/Fc is critical for L-SIGN binding, the inventors expressed wild-type (WT) or mutant forms of the extracellular domain of L-SIGN (residue 78-399 amino acids) fused to the human IgG2 Fc domain (L-SIGN/Fc, FIG. 7A). L-SIGN/Fc but not L-SIGN^{QPD}/Fc bound to spike protein-expressing HEK293T cells. Binding was inhibited by EGTA and by mannan, a β-mannose polymer that blocks interactions with high-mannose N-glycans (FIG. 2F)²⁴. The inventors also treated spike protein-expressing cells with kifunensine, a small molecule that promotes accumulation of high-mannose type N-glycans in the endoplasmic reticulum and Golgi apparatus (FIG. 7B)²⁵. Kifunensine treatment enhanced the binding of spike protein to either L-SIGN/Fc or GNL (FIG. 7C). These data indicate that the EPN motif of L-SIGN recognizes a high-mannose type N-glycan on spike protein in a Ca²⁺-dependent manner (FIG. 7D).

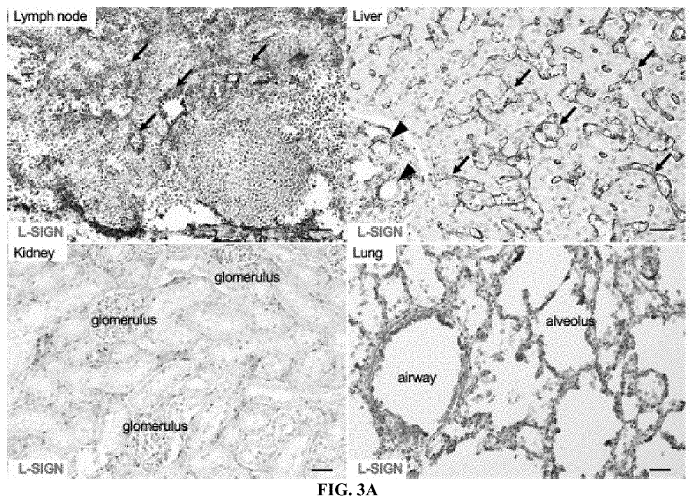

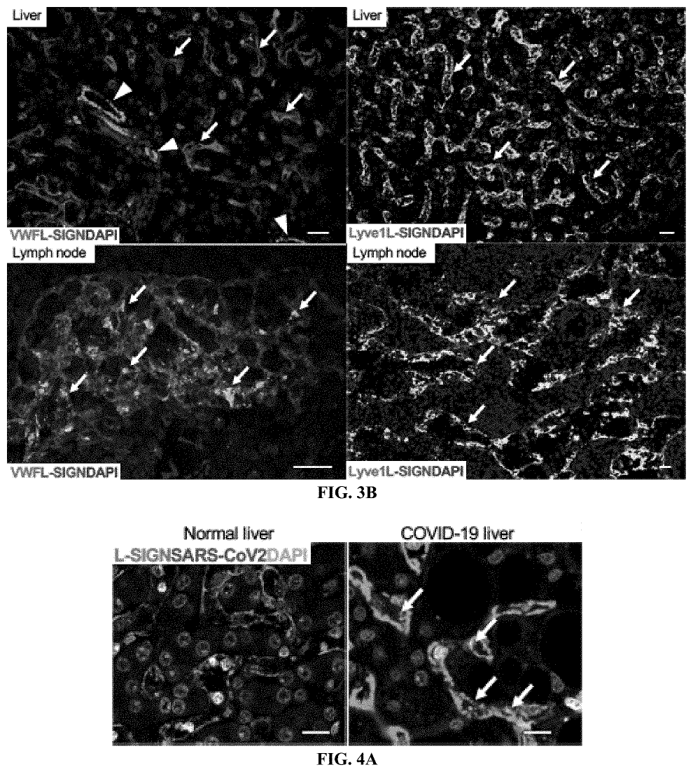

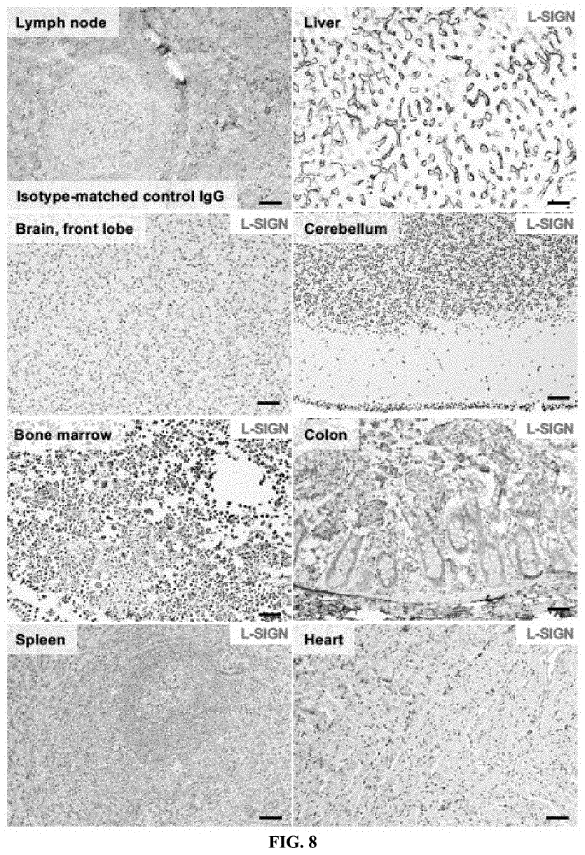

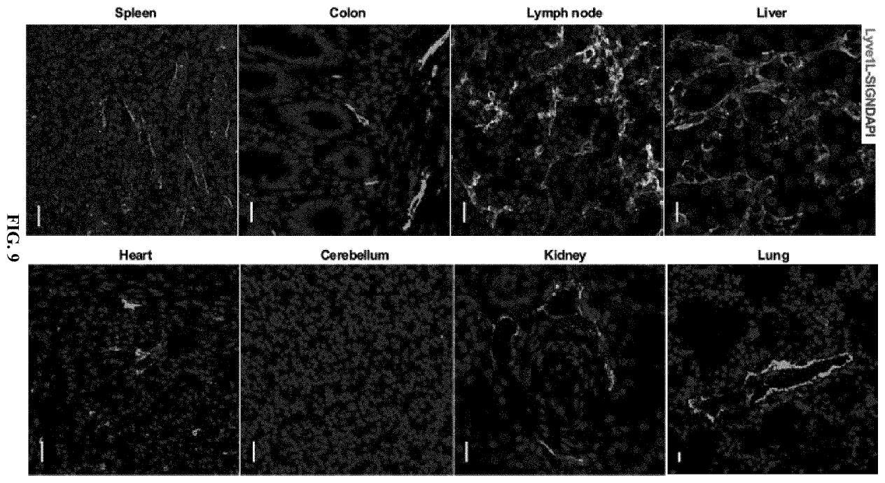

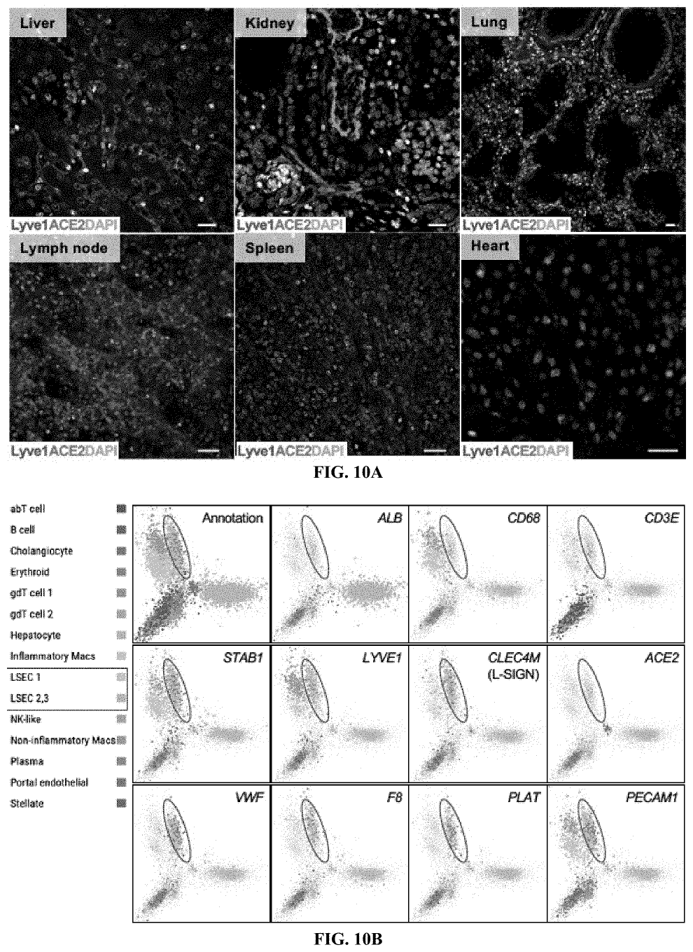

L-SIGN but not ACE2 is highly expressed on LSECs and on lymph node LECs in human biopsy or autopsy tissue samples. Only humans and great apes have genes that encode L-SIGN (FIG. 1J)²⁶. To explore the biological function of interaction between SARS-CoV-2 and L-SIGN, the inventors first profiled the expression of L-SIGN in formalin-fixed paraffin-embedded normal human biopsy as well as autopsy tissue samples²⁷. After antigen retrieval, L-SIGN was detected in LSECs and in LECs in the lymph node with both immunohistochemical and immunofluorescent staining with two different antibodies (Origene #TA810067 and #TA810055) to human L-SIGN (FIG. 3A, 3B). L-SIGN was not detected in other blood vessels such as central vein, portal vein, hepatic artery in the liver or high endothelial venules and arterioles in the lymph nodes. It was also not detected in airway epithelial cells and other cells in the lung or in LECs in other tissues such as intestine, heart, and kidney (FIGS. 8 and 9). Of note, the inventors did not detect ACE2 in LSECs and in lymph node LECs based on immunostaining with two different antibodies (Abcam #ab89111, R&D #AF933) (FIG. 10A). Thus, these data demonstrate that L-SIGN, but not ACE2, is uniquely expressed on LSECs and on LECs in the lymph node.

Flow cytometry was used to measure the expression of L-SIGN and ACE2 in cultured primary or transformed endothelial cell lines. HEK293T cells transfected with either L-SIGN or ACE2 were used as positive controls. Neither receptor was detected on immortalized human umbilical cord vein endothelial cells (HUVECs), a hybrid endothelial cell line EAhy926, or immortalized human dermal microvascular blood endothelial cells (DMECs). A previous mRNA microarray study reported L-SIGN mRNA induction in human endothelial cells upon stimulation with TNFα or IL-4²⁸. However, the inventors did not detect L-SIGN expression in human endothelial cell lines after TNFα or IL-4 challenges (FIG. 11A, 11B). Furthermore, these cells were not susceptible to CoV2-type infection (FIG. 11C, 11D). These data are consistent with tissue staining results that L-SIGN is not expressed by most vascular endothelial cells.

The inventors then examined cultured primary LSECs and LECs. CD31, an endothelial cell marker, was expressed in these cells. However, as previously noted, L-SIGN was not detected (FIG. 11E)²⁹. Moreover, TNFα and IL-6, cytokines elevated in COVID-19 patients, did not stimulate the expression of L-SIGN in these cells (FIG. 11F). LSECs transduced with L-SIGN-FLAG by lentivirus was susceptible to CoV2-type infection (FIG. 11G).

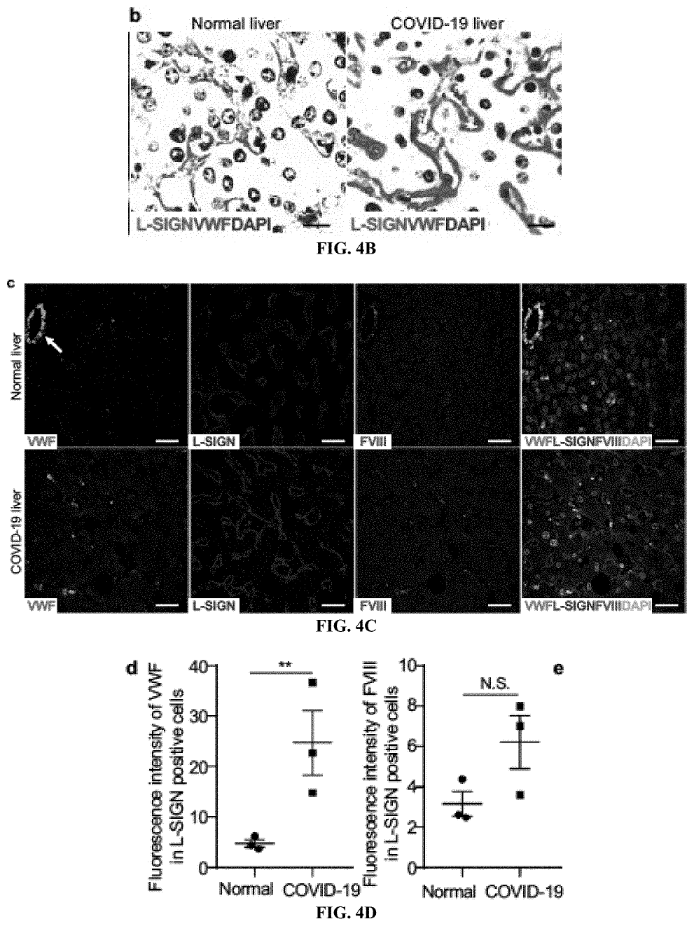

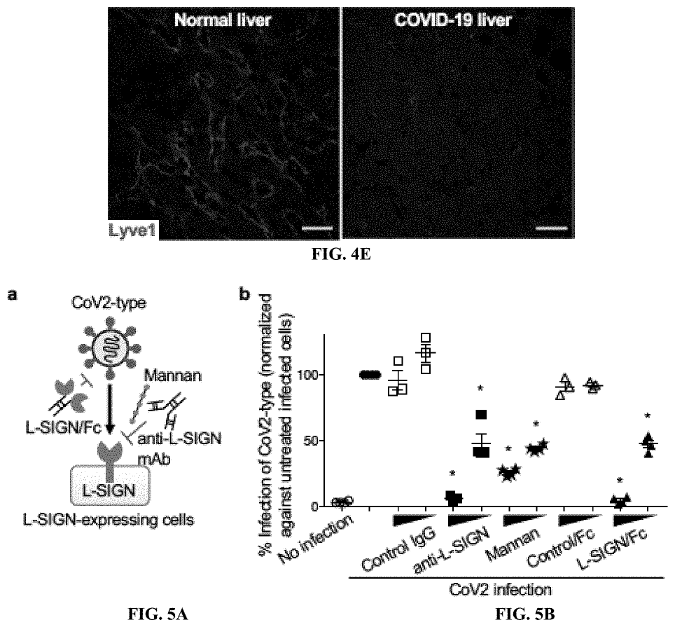

LSECs of COVID-19 patient liver autopsy samples have detectable SARS-CoV-2 and exhibit increased procoagulant activities. To determine the clinical significance of the interaction between SARS-CoV-2 and L-SIGN, the inventors first examined if SARS-CoV-2 was present in LSECs of COVID-19 patients' formalin-fixed paraffin-embedded liver autopsy samples. In comparison with uninfected human liver autopsy samples, the inventors found increased expression of L-SIGN and detected SARS-CoV-2 with an anti-SARS-CoV-2 nucleocapsid antibody (Invitrogen #MA1-7404) in patient LSECs (FIG. 4A). Importantly, significantly increased expression of VWF was detected in the patient LSECs relative to that of uninfected controls (FIG. 4B-4D)^{7, 16, 17}. In addition, expression of FVIII in the patient LSECs relative to that of uninfected controls showed an increased trend even though it was not statistically significant (FIG. 4C, 4D). Co-expression of CLEC4M (encoding L-SIGN) with VWF, F8, and PLAT (encoding key coagulation factors VWF, FVIII, and t-PA) in LSECs was validated by analyzing published single-cell RNA sequencing data from human liver (FIG. 10B)³⁰. Interestingly, Lyve1, a marker for LSECs, was barely detectable in the patient LSECs relative to those of the uninfected controls (FIG. 4E).

Blocking L-SIGN function reduces CoV2-type infection. The inventors treated L-SIGN-expressing HEK293T cells with an antibody to human L-SIGN (anti-L-SIGN, R&D #MAB162), with mannan to block mannose-dependent interactions, or with L-SIGN/Fc recombinant protein. As negative controls, the inventors used isotype-matched control IgG and Fc control protein. Anti-L-SIGN, mannan, or L-SIGN/Fc recombinant protein dose-dependently blocked L-SIGN-mediated CoV2-type infection but not control VSVG-type infection, indicating specific inhibition of L-SIGN-mediated CoV2 infection. CoV2-type infection was not blocked by negative controls (FIGS. 5A, 5B, and FIGS. 7E, 7F). These data demonstrate that infection requires interactions of a mannose glycan on the CoV2-type spike protein with L-SIGN.

These data demonstrate that L-SIGN is as a novel SARS-CoV-2 receptor that interacts with high-mannose N-glycans on the viral spike protein. L-SIGN was highly expressed on LSECs and on LECs in the lymph node of human biopsy or autopsy samples. The inventors detected SARS-CoV-2 in LSECs of COVID-19 autopsy liver samples, which was correlated with procoagulant activities.

L-SIGN is a type II transmembrane protein. The phylogenetic tree suggests that gene duplication of DC-SIGN in the Old World monkey has given rise to L-SIGN (FIG. 1J)²⁶. Only humans and great apes such as gorilla and chimpanzee express L-SIGN (FIG. 1I, 1J). L-SIGN and DC-SIGN are C-type lectin receptors that mediate infections of Ebola virus, Marburg virus, Japanese encephalitis virus, HIV-1, and hepatitis C virus via interactions with viral envelope glycoproteins. SARS-CoV-2 is related to SARS-CoV, which caused the first coronavirus epidemic in 2003³¹. SARS-CoV-2 and SARS-CoV have similar spike proteins. Although the SARS-CoV spike protein binds to both DC-SIGN and L-SIGN, only L-SIGN binding initiates SARS-CoV infection³². L-SIGN binds to the N-glycosylated N-terminus but not to the RBD of the SARS-CoV spike protein. Mutagenesis of N330 in the RBD of SARS-CoV, equivalent to N343 of SARS-CoV-2, does not affect SARS-CoV infection (FIG. 6D)³³. In contrast, the data herein demonstrates that L-SIGN must bind to a high-mannose N-glycan on the spike protein to mediate entry of SARS-CoV-2 into cells. The molecular basis of the difference in the L-SIGN recognition domain of spike protein between SARS-CoV and SARS-CoV-2 remains to be studied³¹.

Previous studies of N-glycans on RBD N343 yielded discrepant results, probably due to different expression systems used to produce recombinant spike protein for glycan profiling^{10, 11}. The inventors' structural analysis indicates that high-mannose N-glycans are common on the spike protein, and the functional assays show that the high-mannose N-glycan is critical for binding to L-SIGN but not to ACE2 (FIG. 2C).

ACE2 has been the only documented entry receptor for SARS-CoV-2^{34, 35}. In human lungs, ACE2 is expressed on alveolar epithelial type II cells, which mediates virus entry and replication that causes acute alveolar epithelial cell injury⁴. Other than the lung, ACE2 is also found in the kidney, small intestine, and heart, which possibly contributes to pathologies in these organs³⁶. Coagulopathy and thrombosis are common complications of patients with severe COVID-19^{5, 7}. Although the causes of coagulopathy in severe COVID-19 are almost certainly multifaceted, including inflammatory responses^{37, 38}, previous studies have demonstrated a role for endothelial cell injury or dysfunction³⁹. However, there is no convincing evidence that ACE2 is expressed in vascular endothelial cells^{39, 40}. As shown herein, ACE2 was not detected in vascular endothelial cells in different human tissues or in multiple human endothelial cell lines from different origins.

Markers of endothelial cell activation such as increased circulating VWF and FVIII have been observed in severe COVID-19 patients in hospital intensive care units, which is correlated with mortality⁷. During severe inflammation, many factors contribute to endothelial cell activation. These data show that interactions of SARS-CoV-2 with L-SIGN on LSECs can directly mediate endothelial injury. Notably, considerably higher levels of L-SIGN on LSECs were detected in liver from patients with COVID-19 than from uninfected controls (FIG. 4A-4C). The COVID-19 patient LSECs lost expression of Lyve-1, a sign of liver inflammation, but exhibited procoagulant activities, suggesting endothelial dysfunction (FIG. 4E)^{7, 41}. LSECs, together with lymph node LECs, are primary sources of FVIII^{16, 18}, which pairs with VWF as essential procoagulants¹⁶. Coagulation changes in the most severe COVID-19 patients are different from typical sepsis-related disseminated intravascular coagulopathy because of less prominent thrombocytopenia and consumptive coagulopathy, suggestive of an unappreciated mechanism⁷. In addition, hyperfibrinolysis (increased D-dimer levels) was prominent in COVID-patients. The inventors found that other than VWF and FVIII, t-PA is also highly expressed in LSECs (FIG. 10B). Activated endothelial cells release t-PA⁴², which could cause the burst of plasmin and subsequent increased D-dimer in COVID-19 patients. Enlarged livers have been noted in COVID-19 patients⁴³. LSECs comprise a significant portion of non-parenchymal cells in the liver, the largest solid organ in humans⁴⁴. L-SIGN-mediated activation of VWF- and FVIII-enriched LSECs may be critical to the pathogenesis of coagulopathy and thrombosis in severe COVID-19 patients⁷.

Multiple animal models of SARS-CoV-2 infection have been reported, ranging from rodents to Old World and New World monkeys^{34, 35, 45, 46, 47}. These animal models display consistent pulmonary inflammatory changes, but none exhibits obvious signs of coagulopathy or thrombosis. This may be explained by the fact that L-SIGN is not expressed in these non-human primates. Other experimental models are needed to identify additional unappreciated factors contribute to COVID-19-related coagulopathy in humans. Based on these results, it is plausible that SARS-CoV-2 infection of LSECs and LECs through L-SIGN leads to endothelial cell activation and secretion of VWF and FVIII into circulation, which may synergize with ACE2-mediated infection to cause coagulopathy in humans^{16, 26, 48}. Indeed, ligand-engagement of L-SIGN is known to trigger mitogen-activated protein kinase cascades^{49, 50}. These data also indicate that caution needs to be taken in extrapolating results from animal models of SARS-CoV-2 infection to the human disease.

Expression of L-SIGN in other tissues or other types of vascular endothelial cells may also contribute to the SARS-CoV-2 infection. However, other than LSECs in the liver and LECs in the lymph node, the inventors did not detect L-SIGN expression in other organs and in vascular endothelium of many types of human tissues, including the lung, despite utilizing two different immunostaining methods, multiple approaches for antigen retrieval, and two independent anti-human L-SIGN antibodies that recognize different epitopes. In addition, the inventors did not detect CoV2-type infection in immortalized HUVECs and DMECs (FIGS. 11A-11G). Therefore, these results do not support L-SIGN as a receptor for SARS-CoV-2 in other tissues or on blood endothelial cells in other organs.

In summary, the present inventors identified L-SIGN as a new, tissue-specific receptor for SARS-CoV-2. By way of explanation, and in no way a limitational of the present invention, these results provide mechanistic insights into the pathogenesis of coagulopathy in severe COVID-19 patients. Importantly, it was found that interactions between SARS-CoV-2 and L-SIGN could be blocked by anti-L-SIGN antibody, by mannan, or by recombinant L-SIGN/Fc protein. These results provide a novel therapeutic option to treat severe COVID-19 infection in general, and viral or virally-induced tissue dysfunction or failure such as tissue damage or tissue injury by decreasing endothelial cell death in the liver or lymph node.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Fauver, J. R. et al. Coast-to-Coast Spread of SARS-CoV-2 during the Early Epidemic in the United States. Cell 181, 990-996 e995 (2020).

2. Hoffmann, M. et al. SARS-CoV-2 Cell Entry Depends on ACE2 and TMPRSS2 and Is Blocked by a Clinically Proven Protease Inhibitor. Cell 181, 271-280 e278 (2020).

3. Shang, J. et al. Cell entry mechanisms of SARS-CoV-2. Proc Natl Acad Sci U S A 117, 11727-11734 (2020).

4. Ziegler, C. G. K. et al. SARS-CoV-2 Receptor ACE2 Is an Interferon-Stimulated Gene in Human Airway Epithelial Cells and Is Detected in Specific Cell Subsets across Tissues. Cell 181, 1016-1035 e1019 (2020).

5. Connors, J. M. & Levy, J. H. COVID-19 and its implications for thrombosis and anticoagulation. Blood 135, 2033-2040 (2020).

6. Ackermann, M. et al. Pulmonary Vascular Endothelialitis, Thrombosis, and Angiogenesis in Covid-19. N Engl J Med 383, 120-128 (2020).

7. Goshua, G. et al. Endotheliopathy in COVID-19-associated coagulopathy: evidence from a single-centre, cross-sectional study. Lancet Haematol 7, e575-e582 (2020).

8. Helms, J. et al. High risk of thrombosis in patients with severe SARS-CoV-2 infection: a multicenter prospective cohort study. Intensive Care Med 46, 1089-1098 (2020).

9. Smith, J. C. et al. Cigarette Smoke Exposure and Inflammatory Signaling Increase the Expression of the SARS-CoV-2 Receptor ACE2 in the Respiratory Tract. Dev Cell 53, 514-529 e513 (2020).

10. Shajahan, A., Supekar, N. T., Gleinich, A. S. & Azadi, P. Deducing the N- and O-glycosylation profile of the spike protein of novel coronavirus SARS-CoV-2. Glycobiology (2020).

11. Watanabe, Y., Allen, J. D., Wrapp, D., McLellan, J. S. & Crispin, M. Site-specific glycan analysis of the SARS-CoV-2 spike. Science 369, 330-333 (2020).

12. Watanabe, Y. et al. Vulnerabilities in coronavirus glycan shields despite extensive glycosylation. Nat Commun 11, 2688 (2020).

13. Marzi, A. et al. DC-SIGN and DC-SIGNR interact with the glycoprotein of Marburg virus and the S protein of severe acute respiratory syndrome coronavirus. J Virol 78, 12090-12095 (2004).

14. Jeffers, S. A. et al. CD209L (L-SIGN) is a receptor for severe acute respiratory syndrome coronavirus. Proc Natl Acad Sci U S A 101, 15748-15753 (2004).

15. Chan, V. S. et al. Homozygous L-SIGN (CLEC4M) plays a protective role in SARS coronavirus infection. Nat Genet 38, 38-46 (2006).

16. Pan, J. et al. Patterns of expression of factor VIII and von Willebrand factor by endothelial cell subsets in vivo. Blood 128, 104-109 (2016).

17. Shahani, T. et al. Human liver sinusoidal endothelial cells but not hepatocytes contain factor VIII. J Thromb Haemost 12, 36-42 (2014).

18. Everett, L. A., Cleuren, A. C., Khoriaty, R. N. & Ginsburg, D. Murine coagulation factor VIII is synthesized in endothelial cells. Blood 123, 3697-3705 (2014).

19. Guo, Y. et al. Structural basis for distinct ligand-binding and targeting properties of the receptors DC-SIGN and DC-SIGNR. Nat Struct Mol Biol 11, 591-598 (2004).

20. Feinberg, H., Mitchell, D. A., Drickamer, K. & Weis, W. I. Structural basis for selective recognition of oligosaccharides by DC-SIGN and DC-SIGNR. Science 294, 2163-2166 (2001).

21. Koppel, E. A., van Gisbergen, K. P., Geijtenbeek, T. B. & van Kooyk, Y. Distinct functions of DC-SIGN and its homologues L-SIGN (DC-SIGNR) and mSIGNR1 in pathogen recognition and immune regulation. Cell Microbiol 7, 157-165 (2005).

22. Shajahan, A., Heiss, C., Ishihara, M. & Azadi, P. Glycomic and glycoproteomic analysis of glycoproteins-a tutorial. Anal Bioanal Chem 409, 4483-4505 (2017).

23. Drickamer, K. Engineering galactose-binding activity into a C-type mannose-binding protein. Nature 360, 183-186 (1992).

24. Gardner, J. P. et al. L-SIGN (CD 209L) is a liver-specific capture receptor for hepatitis C virus. Proc Natl Acad Sci U S A 100, 4498-4503 (2003).

25. Stanley, P., Taniguchi, N. & Aebi, M. N-Glycans. In: rd et al. (eds). Essentials of Glycobiology: Cold Spring Harbor (NY), 2015, pp 99-111.

26. Ortiz, M. et al. The evolutionary history of the CD209 (DC-SIGN) family in humans and non-human primates. Genes Immun 9, 483-492 (2008).

27. Pohlmann, S. et al. DC-SIGNR, a DC-SIGN homologue expressed in endothelial cells, binds to human and simian immunodeficiency viruses and activates infection in trans. Proc Natl Acad Sci U S A 98, 2670-2675 (2001).

28. Sana, T. R., Janatpour, M. J., Sathe, M., McEvoy, L. M. & McClanahan, T. K. Microarray analysis of primary endothelial cells challenged with different inflammatory and immune cytokines. Cytokine 29, 256-269 (2005).

29. Swystun, L. L. et al. The endothelial lectin clearance receptor CLEC4M binds and internalizes factor VIII in a VWF-dependent and independent manner. J Thromb Haemost 17, 681-694 (2019).

30. MacParland, S. A. et al. Single cell RNA sequencing of human liver reveals distinct intrahepatic macrophage populations. Nat Commun 9, 4383 (2018).

31. Walls, A. C. et al. Structure, Function, and Antigenicity of the SARS-CoV-2 Spike Glycoprotein. Cell 181, 281-292 e286 (2020).

32. Yang, Z. Y. et al. pH-dependent entry of severe acute respiratory syndrome coronavirus is mediated by the spike glycoprotein and enhanced by dendritic cell transfer through DC-SIGN. J Virol 78, 5642-5650 (2004).

33. Han, D. P., Lohani, M. & Cho, M. W. Specific asparagine-linked glycosylation sites are critical for DC-SIGN- and L-SIGN-mediated severe acute respiratory syndrome coronavirus entry. J Virol 81, 12029-12039 (2007).

34. Jiang, R. D. et al. Pathogenesis of SARS-CoV-2 in Transgenic Mice Expressing Human Angiotensin-Converting Enzyme 2. Cell 182, 50-58 e58 (2020).

35. Winkler, E. S. et al. SARS-CoV-2 infection of human ACE2-transgenic mice causes severe lung inflammation and impaired function. Nat Immunol (2020).

36. Lee, I. T. et al. Robust ACE2 protein expression localizes to the motile cilia of the respiratory tract epithelia and is not increased by ACE inhibitors or angiotensin receptor blockers. medRxiv (2020).

37. Middleton, E. A. et al. Neutrophil extracellular traps contribute to immunothrombosis in COVID-19 acute respiratory distress syndrome. Blood 136, 1169-1179 (2020).

38. Zuo, Y. et al. Neutrophil extracellular traps in COVID-19. JCI Insight 5 (2020).

39. Varga, Z. et al. Endothelial cell infection and endotheliitis in COVID-19. Lancet 395, 1417-1418 (2020).

40. Teuwen, L. A., Geldhof, V., Pasut, A. & Carmeliet, P. COVID-19: the vasculature unleashed. Nat Rev Immunol 20, 389-391 (2020).

41. Mouta Carreira, C. et al. LYVE-1 is not restricted to the lymph vessels: expression in normal liver blood sinusoids and down-regulation in human liver cancer and cirrhosis. Cancer Res 61, 8079-8084 (2001).

42. Levin, E. G., Marzec, U., Anderson, J. & Harker, L. A. Thrombin stimulates tissue plasminogen activator release from cultured human endothelial cells. J Clin Invest 74, 1988-1995 (1984).

43. Mehta, P. et al. COVID-19: consider cytokine storm syndromes and immunosuppression. Lancet 395, 1033-1034 (2020).

44. Poisson, J. et al. Liver sinusoidal endothelial cells: Physiology and role in liver diseases. J Hepatol 66, 212-227 (2017).

45. Sia, S. F. et al. Pathogenesis and transmission of SARS-CoV-2 in golden hamsters. Nature 583, 834-838 (2020).

46. Deng, W. et al. Primary exposure to SARS-CoV-2 protects against reinfection in rhesus macaques. Science 369, 818-823 (2020).

47. Israelow, B. et al. Mouse model of SARS-CoV-2 reveals inflammatory role of type I interferon signaling. J Exp Med 217 (2020).

48. Ludwig, I. S. et al. Hepatitis C virus targets DC-SIGN and L-SIGN to escape lysosomal degradation. J Virol 78, 8322-8332 (2004).

49. Zhao, L. J., Wang, W., Ren, H. & Qi, Z. T. Interaction of L-SIGN with hepatitis C virus envelope protein E2 up-regulates Raf-MEK-ERK pathway. Cell Biochem Biophys 66, 589-597 (2013).

50. Johnson, T. R., McLellan, J. S. & Graham, B. S. Respiratory syncytial virus glycoprotein G interacts with DC-SIGN and L-SIGN to activate ERK1 and ERK2. J Virol 86, 1339-1347 (2012).

What is claimed is:

1. A method for treating and/or preventing a viral or virally-induced tissue dysfunction or failure in a patient comprising administering an inhibitor of SARS-CoV-2 binding to L-SIGN in an amount effective to treat the viral or virally-induced tissue dysfunction or failure.

2. The method of claim 1, wherein the inhibitor is anti-L-SIGN antibody, a mannan, or a recombinant L-SIGN/Fc protein.

3. The method of claim 1, wherein the inhibitor is formulated in a pharmaceutical composition formulated for oral administration, direct injection, intravenous injection, infusion, local administration, for sustained release, or combinations thereof.

4. The method of claim 1, wherein the inhibitor is at administered to the subject within 8, 12, 16, 24, 36, 48, 60, 72 hours, 4, 5, 6, 7, 8, 9, 10, 11, 12, 13, or 14 days from the onset of symptoms of a viral infection;
the inhibitor is repeatedly administered to the subject at least once per day for at least 3, 4, 5, 6, 7 or more days;
the inhibitor is provided at 20, 50, 100, 200, 500 and 1000 ug/kg/hr; or
the inhibitor is provided over 1, 2, 3, 4, 5, 6, or 7 hours via intravenous infusion.

5. The method of claim 1, wherein the inhibitor reduces damage to a tissue more than 25, 30, 33, 35, 40, 45, or 50% when compared to a non-treated tissue;
the inhibitor reduces a vascular deficit in the liver or lymph node by at least 30, 40, 50, 60, or 70% when compared to a non-treated liver or lymph node;
the inhibitor is injected in situ; or
the inhibitor reduces tissue damage or tissue injury by decreasing endothelial cell death in the liver or lymph node.

6. The method of claim 1, wherein the virally-induced liver dysfunction or liver failure is a diminished microvascular blood flow in the liver caused by an immune response or a cytokine storm.

7. The method of claim 1, wherein the anti-L-SIGN antibody, a mannan, or a recombinant L-SIGN/Fc protein reduces liver tissue scarring in patients that have had a virally induced liver dysfunction or failure when compared to a non-treated liver tissue.

8. The method of claim 1, further comprising the step of identifying the patient as having a SARS-CoV2 infection, determining if the patient is forming one or more blood clots, and providing the inhibitor within 24 hours of the earliest of: detection of the viral infection or blood clots.

9. The method of claim 1, wherein cells in the viral or virally-induced tissue dysfunction or failure to not express ACE2.

10. The method of claim 1, further comprising identifying a patient with a coronavirus infection and damaged liver or lymph node tissue;
imaging a location in a liver or lymph node of a patient with the diminished microvascular blood flow;
obtaining a composition comprising a carrier solution combined with an inhibitor of Sars-CoV-2 binding to L-SIGN; and
treating the liver or lymph node with an effective amount of the inhibitor.

11. A method for treating and/or preventing a viral or virally-induced liver dysfunction or failure comprising at least one of: administering an effective amount of an inhibitor of coronavirus binding to L-SIGN, wherein the amount of inhibitor is sufficient reduce or eliminate the viral or virally-induced liver dysfunction or failure caused by a coronavirus; or administering at least one of: anti-L-SIGN antibody, a mannan, or a recombinant L-SIGN/Fc protein, wherein the viral or virally-induced condition is caused by a coronavirus; or administering an effective amount of an anti-L-SIGN antibody, a mannan, or a recombinant L-SIGN/Fc protein sufficient to reduce or eliminate the damage to liver or lymph node tissue caused by a virally-induced autoimmune response to liver or lymph node tissue.

12. The method of claim 11, wherein the anti-L-SIGN antibody, a mannan, or a recombinant L-SIGN/Fc protein reduces tissue scarring in patients that have had a virally induced liver or lymph node dysfunction or failure when compared to a non-treated tissue.

13. The method of claim 11, further comprising the step of identifying the patient as having a SARS-CoV1, MERS, or SARS-CoV2 infection, determining if the patient is forming one or more blood clots, and providing the anti-L-SIGN antibody, a mannan, or a recombinant L-SIGN/Fc protein within 24 hours of the earliest of: detection of the viral infection or blood clots.

14. The method of claim 11, wherein cells in the viral or virally-induced tissue dysfunction or failure to not express ACE2.

15. The method of claim 11, wherein the coronavirus is SARS-CoV2.

16. A method for treating and/or preventing a viral or virally-induced formation of blood clots or epithelial cell damage in the liver or lymph node in a patient comprising:
identifying the patient as having a SARS-CoV2 infection;
determining if the patient is forming one or more blood clots or has or epithelial cell damage in the liver or lymph node;
administering at least one of: an anti-L-SIGN antibody, a mannan, or a recombinant L-SIGN/Fc protein in an amount effective to treat the viral or virally-induced blood clot formation or epithelial cell damage, wherein the anti-L-SIGN antibody, a mannan, or a recombinant L-SIGN/Fc protein is administered within 24 hours of the earliest of: detection of the viral infection, detection of blood clots, or detection of or epithelial cell damage, wherein the anti-L-SIGN antibody, a mannan, or a recombinant L-SIGN/Fc protein prevents or reduces the effect of blood clots or epithelial cell damage in the liver or lymph node.
